(19) United States
(12) Patent Application Publication
(10) Pub. No.: US 2016/0374636 A1
(43) Pub. Date: Dec. 29, 2016

(54) RADIOGRAPHIC DEVICE

(71) Applicant: SHIMADZU CORPORATION, KYOTO-SHI (JP)

(72) Inventor: RYO TAKEDA, KYOTO-SHI (JP)

(73) Assignee: SHIMADZU CORPORATION, KYOTO-SHI (JP)

(21) Appl. No.: 14/754,056

(22) Filed: Jun. 29, 2015

(51) Int. Cl. A61B 6/00

(52) U.S. Cl. CPC A61B 6/5282; A61B 6/487

(57) ABSTRACT

A radiographic device in which the scatter radiation and the grid foil can be removed without preparing the number of patterns corresponding to the position shift despite the occurrence of the position shift of a scatter radiation removal module relative to a radiation detection module. A foil shade integration value image A_SHsum (g, y, a), I_SHsum (g, y,a), which is normalized to the sum of absorption rates of a direct radiation relative to individual grid foils on a shooting with and without the subject, are respectively calculated and a distance function D (c) based on these absorption rates calculated. Given the foil shade integration value image A_SHsum (g, y, a) selected from a plurality of the foil shade integration value images A_SHsum (g, y, a), wherein the distance function D (c) becomes minimum, the scatter x-ray and the grid foil can be removed without preparing a number of patterns corresponding position shifts despite an occurrence of the position shift of the grid relative to the detector.

### CROSS REFERENCE TO RELATED APPLICATIONS

This application relates to JP Ser. No. 2013-000487 filed Jan. 7, 2013, published as JP Pub. 2014-13154 on Jul. 17, 2014, the entire contents of which are incorporated herein fully by reference.

### FIGURE SELECTED FOR PUBLICATION

FIG. 1

### BACKGROUND OF THE INVENTION

Field of the Invention

The present invention relates to a radiographic device to obtain a radiographic image and particularly relates to a scatter radiation removal technology by using a scatter radiation removal means.

Description of the Related Art

This type of the radiographic device is applied for a medical radiographic image diagnosis device and an industrial radiographic image inspection device. The industrial radiation image inspection device may include a nondestructive inspection device and an X-ray inspection device to inspect the integration circuits on the substrate and the solder connection element and so forth. The medical radiographic image diagnosis device may include a medical X-ray fluoroscopic device to conduct the X-ray fluoroscopy for a human subject. Hereafter, the inventor illustrates the present invention based on e.g., X-ray as the radiation and e.g., a medical X-ray fluoroscopic device as the radiographic device.

With regard to the medical X-ray fluoroscopic device, a scatter X-ray takes place when an X-ray transmits the human subject. The scatter X-ray from the subject causes lowering the quality of the image. Then, a grid (scatter radiation removal means or module) consisting of a grid foil to absorb the scatter radiation and an intermediate layer through which the radiation transmits, which are alternatively set, is employed to suppress lowering the quality of the image due to the scatter X-ray. The grid foil is made of e.g. lead that absorbs a radiation represented by X-ray and the intermediate layer is made of an intermediate material (spacer) including aluminum, an organic substance and graphite through which the radiation represented by X-ray transmits. When X-ray, however, transmits the intermediate layer, X-ray (the direct X-ray) other than the scatter X-ray is also absorbed by the intermediate material (spacer).Then, an air-grid assuredly transmits the X-ray (direct X-ray) other than the scatter X-ray is employed as a grid to date, wherein the intermediate layer is an empty space.

Meantime, the foil shade due to the grid foil is projected in the X-ray image (shooting image) as a fine lattice pattern and overlapped thereon in the part in which the direct X-ray is blocked by the grid foil. A flat panel type X-ray detector (FPD: Flat Panel Detector) is recently applied as an X-ray detector, and the FPD increases the space resolution of the shooting image and sensitivity therefor so that the utilization thereof is rapidly increasing. On the other hand, the higher space resolution and sensitivity of the X-ray detector, the clearer the foil shade becomes, by which the diagnostic imaging is interfered. Then, a method for removing the foil shade due to the grid foil, in which the image is processed by utilizing the frequency conversion, is employed to remove the foil shade (e.g., refer to Patent Document 1).

On the other hand, a synchronous type grid is disclosed, wherein grid foils are in place as the foil shade is projected in the integral multiplicity of the distance (pixel pitch) between the pixels of the shooting image (e.g., refer to Patent Document 2). The above air-grid is employed as a synchronized grid. As described above, when the air-grid is employed as the synchronized grid, no spacer such as aluminum, an organic substance or graphite is employed. Accordingly, the detection efficiency of the direct X-ray can increase. Whereas, the grid foil cannot be supported by a spacer from a manufacturing standpoint therefor and a structure standpoint thereof so that more or less the deformation of the linear grid foil takes place, by which the deformation of the foil shade more or less may take place. Therefore, according to the method for removing the shade of the grid foil utilizing the above frequency conversion, the foil shade having the lattice pattern cannot be sufficiently removed.

Then, the method for removing the foil shade as for the synchronized grid is proposed (e.g., refer to Patent Document 3). According to the grid foil removal method disclosed in Patent Document 3, with regards to the X-ray fluoroscopic device having a synchronized grid, an image displaying the direct transmission coefficient (“direct line transmittance” in Patent Document 3) and an image displaying the scatter line transmittance are calculated based on the images, which are an X-ray image (“air-image”) taken without a subject that is not set in advance, and a phantom image taken with a subject that is an acrylic board. Next, the desired X-ray image of the subject (simply “subject image”) is taken.

Then, the simultaneous equation consisting of each pixel value of the image of the direct transmission coefficient, the scatter transmission coefficient and the subject image is solved to calculate the direct X-ray image of the subject and the scatter X-ray image. The direct X-ray image represents the immediate direct X-ray image that has been transmitting the subject and entering into the synchronized grid.

However, for example, a C-arm X-ray fluoroscopic shooting device, which is curved like C-shape because one end holds the X-ray tube and the other end holds the FPD, may have a small deflection in the C-arm along with the rotation and movement of C-arm because of the weight of the X-ray tube and the FPD. The position of the X-ray tube focal point on the FPD slightly shifts (approximately 2 mm at a maximum) because of such deflection.

Unfortunately, given the position of the X-ray tube focal point shifts, the image of the direct line transmission coefficient and the image of the scatter line transmission coefficient will subtly change against the prospective image of the direct line transmission coefficient and the prospective image of the scatter line transmission coefficient. Consequently, even if the simultaneous equation consisting of each pixel value of the image of the pre-measured direct line transmission coefficient and the image of the pre-measured scatter line transmission coefficient and the subject image of which the X-ray tube focal point is shifted is solved, not only the shade of the grid foil cannot be sufficiently removed but also an artifact (false image) will appear. Accordingly, the foil shade removal method disclosed in Patent Document 3 cannot be applied to the X-ray fluoroscopic device in which the position of the X-ray tube focal point on the FPD changes.

Whereas, the method capable of removing the foil shade is disclosed, despite the change of the position of the X-ray tube focal point (e.g., Patent Document 4). The procedure according to Patent Document 4 includes the steps of (1)-(4).

Specifically, (1) a plurality of air-images (X-ray image without a subject) are taken in advance by gradually changing the X-ray tube focal point along with the parallel plane to the detection plane of the FPD and in the configuration direction of the grid foil (in the direction perpendicular to the extending direction of the grid foil.) (2) After shooting the subject image, the subject line data is selected from a line in the subject image and then the same numbers of air-line data are generated from a plurality of air-images at the lines corresponding to the selected subject line data. (3) Each correlation value as to the grid foil shade is calculated from one subject line data and a plurality of air-line data. (4) The air-image having the maximum correlation value is selected so that the above grid foil shade removal calculation can be conducted.

The correlation value calculated according to the above steps (1)-(4) takes the maximum value when both the position of the X-ray tube focal point at which the subject line data is taken and the position of the X-ray tube focal point are the same and the larger the difference of the positions of the X-ray tube focal points, the smaller the value is. Accordingly, the air-line data taken at the same position or the nearest position, i.e., the corresponding air-image can be selected by comparing the correlation values.

By using this procedure, in the case of C-arm X-ray fluoroscopic device, even when the subtle difference (approximately 2 mm as maximum) of the X-ray tube focal point on the FPD, which cannot be controlled on shooting the subject, the air-image of each X-ray tube focal point moved in the appropriate plural intervals in advance is taken and stored respectively, the most appropriate real-time air-image after shooting the subject can be selected so that the grid foil shade can be removed without producing an artifact.

Further, when the image of direct line transmission coefficient of the subject image and the air-image, i.e., the direct X-ray radiation amount without the grid, is “1.0”, the method to obtain the direct X-ray transmission rate (so-called CP-value) in one pixel is disclosed (e.g., Patent Document 5).

### PRIOR ART DOCUMENTS

### Patent Document

### Patent Document 1: JP Patent Published 2000-83951

### Patent Document 2: JP Patent Published 2002-257939

### Patent Document 3: JP Patent Published 2009-172184

### Patent Document 4: JP Patent Published 2011-101686

### Patent Document 5: JP Patent Published 2010-213902

### ASPECTS AND SUMMARY OF THE INVENTION

### Problems to be Solved by the Invention

However, in the case of Patent Document 4, there is the following limitation.

Specifically, the limitation is that the positional relationship between the FPD and the grid must be the same while both the air-image shooting and the subject shooting. Specifically, the positional relationship between the FPD and the grid must be maintained while the air-image shooting when the subject is being shot. On the other hand, the grid position to the FPD may be shifted in the device such as a C-arm X-ray fluoroscopic device with revolving or movement of the C-arm holding the X-ray tube and the FPD.

If the grid position to the FPD shifts even if the positions of the X-ray focal point on both the air-image shooting and the subject shooting are the same, the profile of the grid foil shade changes as shown in the lower area of FIGS. 7A, 7B. Accordingly, the relationship between the X-ray focal point and the line data (the profile of the grid foil shade) will no longer exist. As results, according to the method disclosed in Patent Document 4, the best air-image cannot be selected because given the X-ray focal points on shooting the air-image and shooting the subject are the same each other, the line data (profiles of the grid foil shade) are the same.

Accordingly, in order to apply the method disclosed in Patent Document 4, not only the position shift of the X-ray tube focal point on the FPD but also the position shift of the grid on the FPD must be considered. Specifically, when N_{A} is the pattern number of the position shifts of the X-ray tube focal point on the FPD and N_{B} is the pattern number of the position shifts of the grid on the FPD, the number of the air-images of N_{A}×N_{B} must be taken in advance. Therefore, increase of the shooting number of the air-images and increase of the memory area to store the air-images are unavoidable. Accordingly, it would be a problem to be solved, in which the number of patterns corresponding to the position shifts of the grid on the FPD increases.

Considering the above situation, the present invention was completed, in which the purpose of the present invention is to provide an radiographic device in which the scatter radiation and the grid foil can be removed without preparing the number of patterns corresponding to the certain shift even despite occurrence of the position shift of the scatter radiation removal means relative to the radiation detection means.

### Means for Solving the Problem

The inventor of the present invention studied extensively to solve the above problem and found the following solutions.

Specifically, according to Patent Document 4, it is the method for which the pattern of the grid foil shade (profile of the grid foil shade) on the air-image shooting (shooting without the subject) and the subject shooting are compared each other to calculate the correlation value, and the sum of square of pixel values as to all grid foils as described in paragraphs [0064]-[0066]. Given such sum of square is obtained as the correlation value, the correlation level as to each grid foil can be uncertain when the position of the grid (scatter radiation removal means) relative to the FPD (radiation detection means) changes due to the shift thereof.

Then, the inventor of the present invention had focused on the individual grid foil. Specifically, an absorption rate of direct radiation relative to the individual grid foils on the air-image shooting (shooting without the subject) and the subject shooting is respectively calculated to calculate the distance function based on these absorption rates. The distance function indicates the correlation level between the radiation image without the subject and the radiation image of the subject and also is the parameter (physical value) indicating the distance of the grid foil from the ideal value. Given the absorption rate of the direct radiation of each grid foil relative to a plurality of the radiation images without the subject based on the distance function is selected, the absorption rate can be selected from only a number of conventional patterns without preparing a number of patterns corresponding to the certain position shift despite the position shift of the grid (scatter radiation removal means) relative to the FPD (radiation detection means), and the inventor have found that the scatter radiation and the grid foil based on the selected absorption rate and the radiation image of the subject could be removed.

The present invention based on the finding constitutes at least the following features and aspects.

Specifically, a radiographic device of the present invention is the radiographic device to obtain a radiographic image, a radiation source that irradiates a radiation, a radiation detection means, wherein the radiation detection elements to detect the irradiated radiation are installed lengthwise and breadthwise, a scatter radiation removal means, wherein grid foils that absorbs the scatter radiation are set in the detection side of the radiation detection means and installed side-by-side parallel in at least one of either lengthwise or breadthwise direction of the radiation detection elements, a image generation means that generates the radiation image based on the radiation detection signal by the radiation detection means, and further, the radiographic device comprises; a first absorption rate calculation means that calculates the absorption rate of the direct radiation of each grid foil as the first absorption rate relative to a plurality of radiation images without a subject based on the radiation detection signals detected without a subject that would be the subject set between said radiation source and said radiation detection means, the second absorption rate calculation means that calculates the absorption rate of the direct radiation of each grid foil as the second absorption rate based on the radiation detection signals detected with a subject that would be the subject set between the radiation source and the radiation detection means, a distance function calculation means that calculates the distance function indicating the correlation level between the radiation image without the subject and the radiation image with the subject and also the distance of the grid foil from the ideal value, based on the first absorption rate calculated by the first absorption calculation means and the second absorption rate calculated by the second absorption calculation means, a selection means that selects the first absorption rate from a plurality of the first absorption rates, wherein the distance function calculated by the distance function calculation means becomes minimum, and an image processing means that executes the image processing to obtain the radiation image in which the foil shade is removed by removing the foil shade due to the grid foil of the radiation image of the subject and calculating the component of the direct radiation of the subject based on the first absorption rate selected by the selection means and the radiation image of the subject.

### [Action and Effects]

According to the radiographic device of the present invention, the first absorption rate calculation means calculates the absorption rate of the direct radiation of each grid foil, as the first absorption rate, relative to a plurality of radiation images without a subject based on the radiation detection signals detected without a subject that would be the subject set between the radiation source and the radiation detection means.

On the other hand, the second absorption rate calculation means calculates the absorption rate of the direct radiation of each grid foil, as the second absorption rate, relative to radiation images with a subject based on the radiation detection signals detected with a subject that would be the subject set between the radiation source and the radiation detection means.

Then, the distance function calculation means calculates the distance function based on the first absorption rate calculated by the first absorption rate calculation means and the second absorption rate calculated by the second absorption rate calculation means. As described above, the distance function indicates the correlation level between the radiation image without the subject and the radiation image of the subject and also is the parameter indicating the distance of the grid foil from the ideal value.

The selection means selects the first absorption rate from a plurality of the first absorption rates, wherein the distance function calculated by the distance function calculation means becomes a minimum. Specifically, the larger correlation between the radiation image without the subject and the radiation image with the subject, the closer the distance to the ideal value and the distance function closes to “0”. Accordingly, the first absorption rate providing the minimum distance function is selected as the optimum absorption rate, Then, an image processing means that executes the image processing to obtain the radiation image in which the foil shade is removed by removing the foil shade due to the grid foil of the radiation image of the subject and calculating the component of the direct radiation of the subject based on the first absorption rate selected by the selection means and the radiation image of the subject.

Accordingly, given a plurality of the first absorption rates based on the distance function is selected, the scatter radiation and the grid foil can be removed without preparing a number of patterns corresponding to the certain position shift despite the position shift of the scatter radiation removal means relative to the radiation detection means.

According to the radiographic device of the present invention, the first absorption rate calculation means, the second absorption rate calculation means and the distance function calculation means specifically calculate as follows. Specifically, the first absorption rate calculation means calculates the direct radiation absorption rate of each grid foil relative to each pixel of the plural radiation images without a subject and the sum of each grid foil absorption rate every pixel unit in-between thereof as one unit from a least affected pixel to another least affected pixel by the foil shade of each grid foil among pixels of the plural radiation images without the subject. The second absorption rate calculation means calculates, almost as well as the first absorption rate calculation means, the direct radiation absorption rate of each grid foil relative to each pixel of the plural radiation images with a subject and the sum of each grid foil absorption rate every pixel unit in-between thereof as one unit from a pixel to another pixel least affected by the foil shade of each grid foil among pixels of the plural radiation images with the subject. The distance function calculation means calculates the distance function based on the sum of absorption rates of each grid foil calculated by the first absorption rate calculation means and the sum of second absorption rates of each grid foil calculated by the second absorption rate calculation means.

It is preferable that the radiographic device according to the present invention has the following variation rate calculation means. Specifically, the variation rate calculation means calculates the variation rate of the radiation image with the subject related to the radiation image without the subject calculates based on the direct radiation absorption rate of each grid foil relative to the plural radiation images without the subject, calculated by the first absorption rate calculation means, and the direct radiation absorption rate of each grid foil relative to the radiation images with the subject, calculated by the second absorption rate calculation means. The variation rate can be ideally “1”. The distance function calculation means calculates the distance function based on the variation rate calculated by the variation rate calculation means so that the larger correlation between the radiation image without the subject and the radiation image with the subject, the closer the variation rate to the ideal value and the distance function closes to “0”. Accordingly, the distance function will be able to be calculated by using the variation rate.

Particularly, when the distance function is calculated by using the variation rate, it is useful when the scatter radiation removal means constituting the grid foils arrayed parallel to only one direction (e.g., line direction) of either lengthwise or breadthwise direction of radiation detection element. According to the scatter radiation removal means having such structure, the grid foils would not crossover in the grid foils extending direction (when the grid foils are arrayed parallel to the line direction, the grid foils extending direction is the column direction) so that the statistical error (i.e., noise) may take place in the extending direction. Then, in order to lower the impact due to the noise of the image, the add-value calculation means adds the variation rate calculated by the variation rate calculation means along the extending direction of the grid foils or conducts an additional averaging (so called “add-average”), which is adding and then averaging, to calculate the add-value due to the adding or the additional averaging. The distance function calculation means calculates a distance function based on the add-value calculated by the add-value calculation means so that the impact due to the noise of the image may effectively lower.

It is preferable that the radiographic device of the present invention includes the first absorption rate memorizing means that writes and memorizes the first absorption rate calculated by the first absorption rate calculation means and conducts a variety of operations by reading out and employing the first absorption rate memorized in the first absorption rate memorizing means thereof. Given the radiation image without the subject is written and memorized, the first absorption rate must be operated by reading out the radiation image without the subject every calculation of the first absorption rate by the first absorption rate calculation means, but if the first absorption rate calculated in advance is written and memorized, the number of operations can be effectively decreased. Further, the memory area, in which the radiation image without the subject is written and memorized, requires a size for entire pixels, but the size of the memory area, in which the first absorption rate is written and memorized, can be decreased to the size only for the grid foils so that the size of the memory area of the first absorption rate memorizing means can be effectively decreased.

### Effects of the Invention

According to the radiographic device of the present invention, an absorption rate of direct radiation relative to the individual grid foils on the shooting without the subject and the shooting with the subject is respectively calculated to calculate the distance function based on these absorption rates. The distance function indicates the correlation level between the radiation image without the subject and the radiation image of the subject and also is the parameter indicating the distance of the grid foil from the ideal value. Given the absorption rate (first absorption rate) of the direct radiation of each grid foil relative to the radiation images without the subject, when the distance function becomes minimum, is selected from a plurality of the first absorption rates, the absorption rate can be selected from only a number of conventional patterns without preparing a number of patterns corresponding to the certain position shift despite the position shift of the scatter radiation removal means relative to the radiation detection means and the scatter radiation and the grid foil can be removed.

The above and other aspects, features and advantages of the present invention will become apparent from the following description read in conjunction with the accompanying drawings, in which like reference numerals designate the same elements.

### BRIEF DESCRIPTION OF THE DRAWINGS

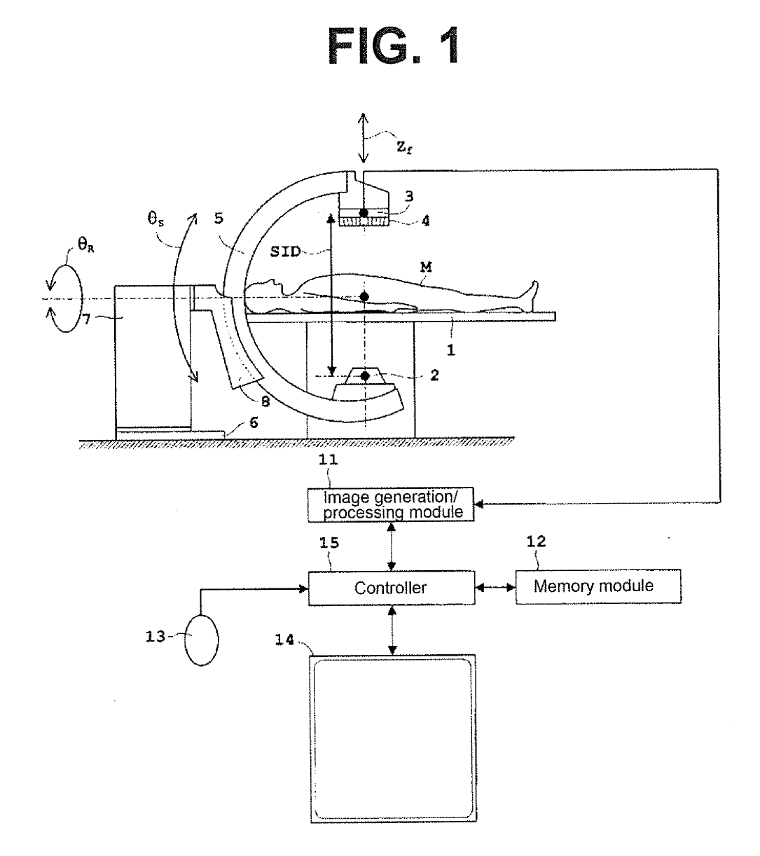

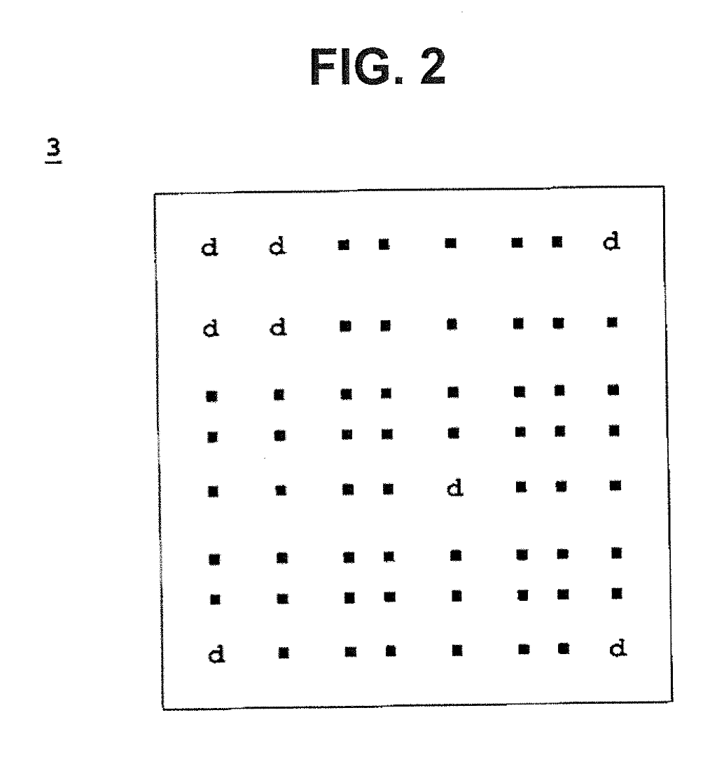

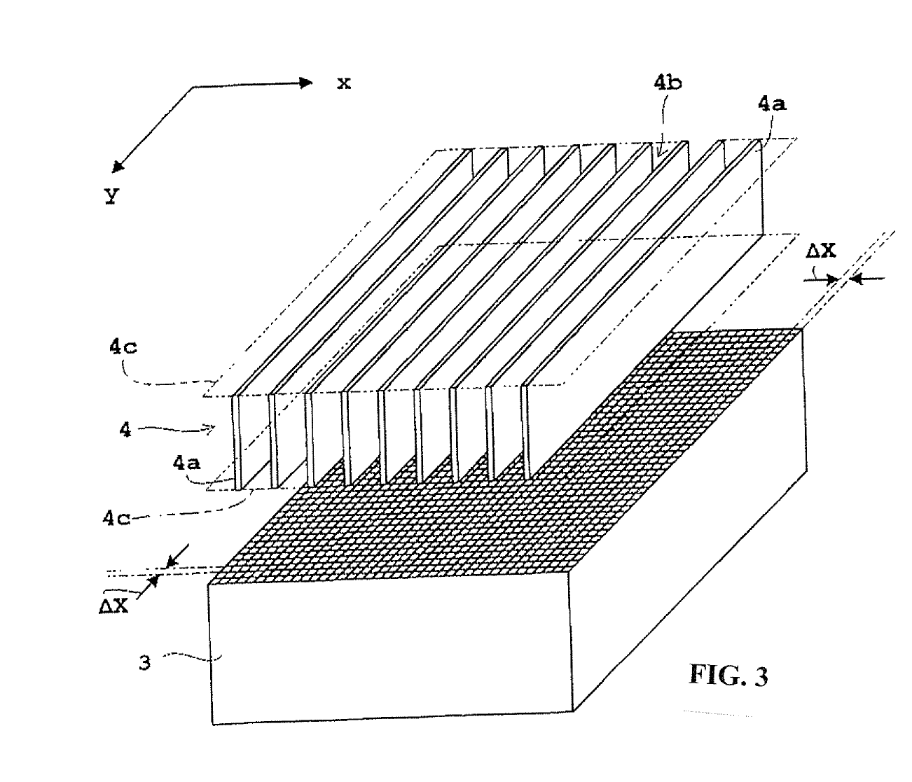

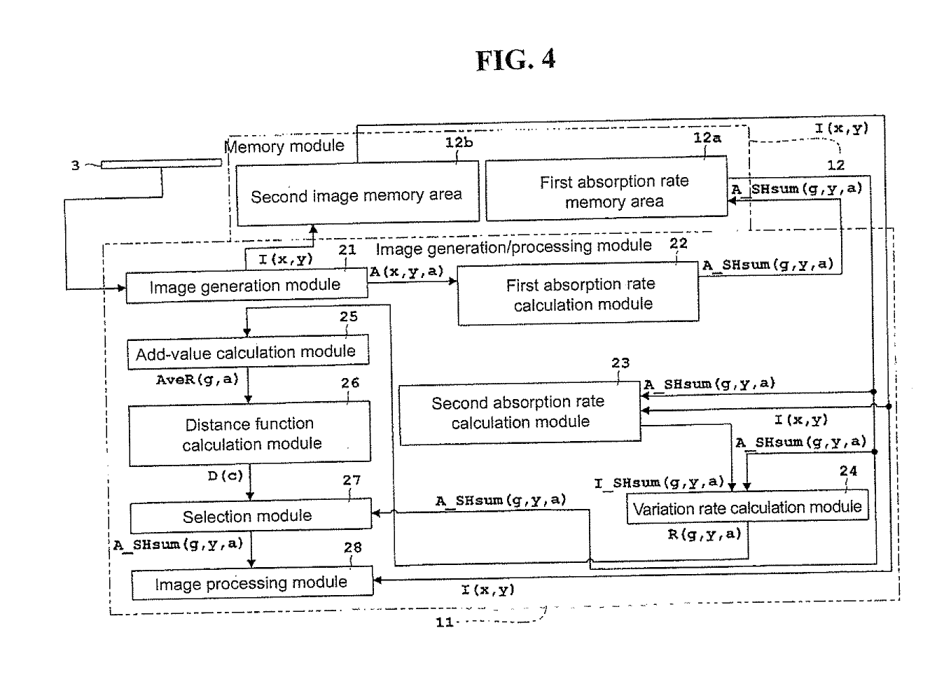

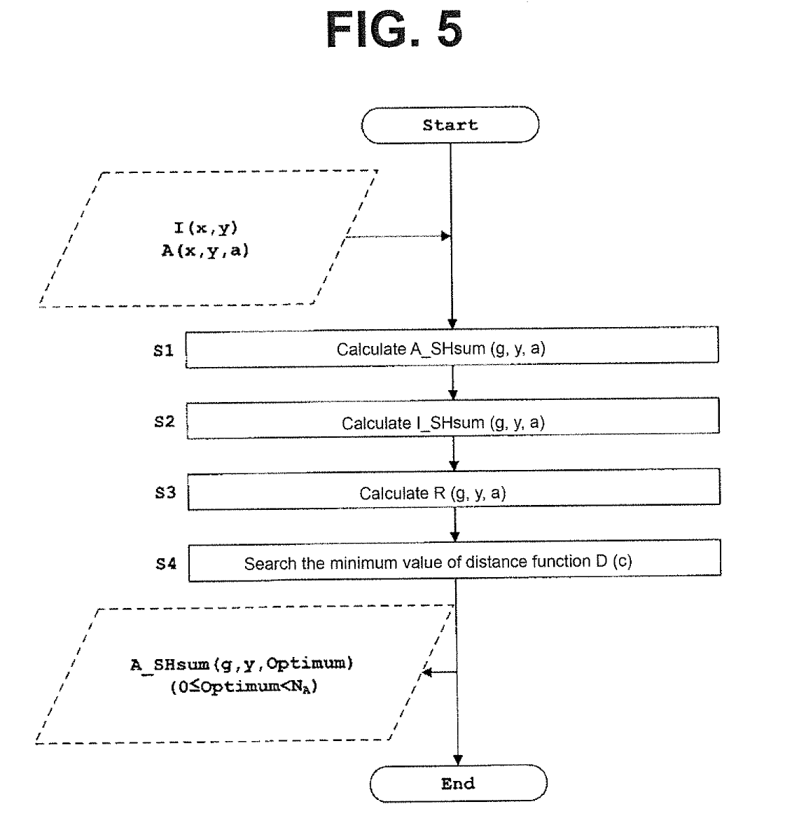

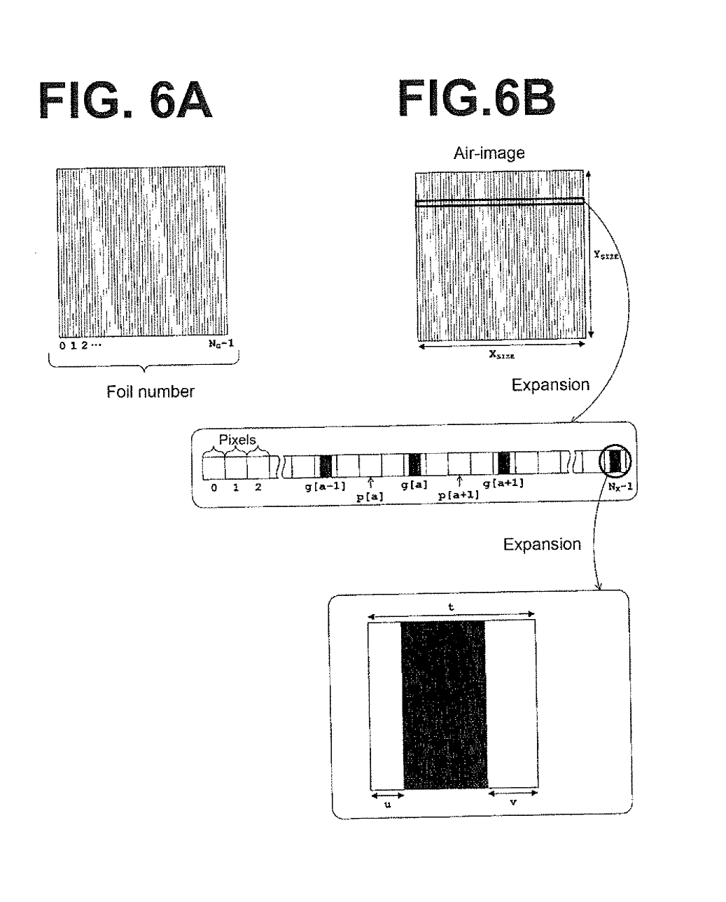

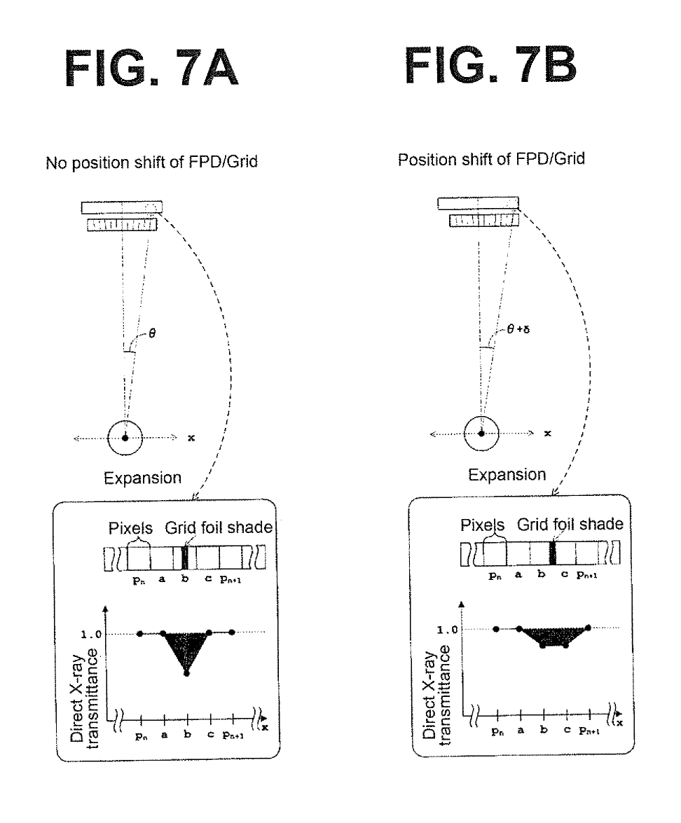

FIG. 1 is a schematic view and a block diagram illustrating the structure of an X-ray radiographic device of Embodiment.

FIG. 2 is a schematic view illustrating the detection plane of a flat panel type X-ray detector (FPD).

FIG. 3 is a schematic view illustrating a grid.

FIG. 4 is a block diagram illustrating specific image generation/processing module of Embodiment.

FIG. 5 is a flow diagram of a series of image processing.

FIG. 6A is a schematic view illustrating one embodiment of the foil shade.

FIG. 6B is a schematic view illustrating one embodiment of the peak pixel coordinate/positional relationship of foil shade coordinate, a CP-value (direct X-ray transmission rate) and an amount of foil shades.

FIG. 7A is a schematic view illustrating a variation of the grid foil shade and the profile of the direct X-ray transmission rate due to no position shift of the FPD/the grid.

FIG. 7B is a schematic view illustrating a variation of the grid foil shade and the profile of the direct X-ray transmission rate due to the position shift of the FPD/grid.

### DETAILED DESCRIPTION OF THE PREFERRED EMBODIMENTS

Reference will now be made in detail to embodiments of the invention. Wherever possible, same or similar reference numerals are used in the drawings and the description to refer to the same or like parts or steps. The drawings are in simplified form and are not to precise scale. The word ‘couple’ and similar terms do not necessarily denote direct and immediate connections, but also include connections through intermediate elements or devices. For purposes of convenience and clarity only, directional (up/down, etc.) or motional (forward/back, etc.) terms may be used with respect to the drawings. These and similar directional terms should not be construed to limit the scope in any manner. It will also be understood that other embodiments may be utilized without departing from the scope of the present invention, and that the detailed description is not to be taken in a limiting sense, and that elements may be differently positioned, or otherwise noted as in the appended claims without requirements of the written description being required thereto.

Various operations may be described as multiple discrete operations in turn, in a manner that may be helpful in understanding embodiments of the present invention; however, the order of description should not be construed to imply that these operations are order dependent.

### Embodiment

Referring to Figures, the inventor illustrates Embodiments of the present invention.

FIG. 1 is a schematic structural view and a block diagram of an X-ray radiographic device of Embodiment. FIG. 2 is a schematic view of the detection plane of a flat panel type X-ray detector (FPD) and FIG. 3 is a schematic view of the grid. In the present Embodiment, the inventor illustrates referring to e.g., X-ray as a radiation, in which, for this instance, the radiographic device is a C-arm X-ray fluoroscopic device having C-arm performed in e.g., an device applied in a cardiovascular diagnosis (CVS: cardiovascular system) is illustrated. Further, as a scatter radiation removal means, the inventor illustrates Embodiment of the air grid that is a focused grid in which the grid foils are in place along the radiation line connecting focal points of the X-ray tube, in which the interlayer is an air gap. The air grid is also a synchronous type grid, wherein grid foils are in place as the foil shade is projected in the integral multiplicity of the distance (pixel pitch) between the pixels of the shooting image.

The X-ray radiographic device according to the present Embodiment, referring to FIG. 1, comprises the table 1 on which the subject is loaded, an X-ray tube 2 radiates X-ray, a flat panel detector (hereafter FPD) 3 and a grid 4 in which a grid foil 4a (referring FIG. 3 and so on) that absorbs the scatter X-ray is installed side-by-side parallel at the detection side of the FPD 3 thereof in either one of lengthwise or breadthwise direction (line direction of the present Embodiment) of the X-ray detection elements d (referring FIG. 2). The X-ray tube 2 is corresponding to the radiation source of the present invention, the flat panel type X-ray detector (FPD) 3 is corresponding to the radiation detection means of the present invention, and the grid 4 is corresponding to the scatter radiation removal means.

In addition, the X-ray radiographic device further includes a C-arm 5 that holds the X-ray tube 2 at one end and the FPD and the grid 4 together at the other end. Referring to FIG. 1, the C-arm 5 is formed as in a curve shape (curved like C shape) in the body axis direction of the subject M. The C-arm 5 revolves around the revolving center axis perpendicular to the body axis of the subject M along the C-arm per se (arm send θ_{S} in FIG. 1) so that the X-ray tube 2, the FPD 3 and the grid 4, which are held by the C-arm 5, can also revolve in the same direction. Further, the C-arm 5 revolves around the revolving center axis of the body axis of the subject M (arm revolving θ_{R} in FIG. 1) so that the X-ray tube 2, the FPD 3 and the grid 4, which are held by the C-arm 5, can also revolve in the same direction.

Specifically, the C-arm 5 is held on the base 6 fixed on the floor by the support column 7 and the arm holding module 8. The support column 7 relative to the base 6 can revolve around the axis center of the perpendicular axis so that the entire C-arm held by the support column 7, the X-ray tube 2, the FPD 3 and the grid 4 can revolve in the same direction because of this revolving. Further, the arm holding module 8 relative to the support column is held to be able to revolve around the revolving center axis of the body axis of the subject M so that the entire C-arm 5 hold by the arm hold element 8, the X-ray tube 2, the FPD 32 and the grid 4 can also revolve in the same direction. Further, the C-arm 5 relative to the arm hold element 8 is hold to be able to revolve orthogonally around the revolving center axis of the body axis of the subject M so that the entire C-arm 5, the X-ray tube 2, the FPD 3 and the grid 4 can also revolve in the same direction.

Further, the FPD 3 structurally can be close/away along the X-ray radiation axis connecting the X-ray tube 2 and the FPD 3, or can be close/away in the focused line direction (arrangement direction of the grid foil) orthogonal to the radiation axis. It can be close/away along the radiation axis so that SID can change every Zf. Further, SID is the distance (SID: Source Image Distance) from the focus position of the perpendicular direction to the FPD 3 when the perpendicular line is drawn from the focus position of the X-ray tube 2 to the FPD 3. In addition, even if the positional relationship among the X-ray tube 2, the FPD 3 and the grid 4 must be conditionally constant, a subtle deflection relative to the C-arm 5 takes place along the movement or revolving of the C-arm 5 and in case the position shift of the positional relationship among the X-ray tube 2, the FPD 3 and the grid 4 takes place.

Further, the X-ray radiographic device comprises; an image generation/processing module 11 that generates the X-ray image based on the X-ray detection signal detected by the FPD 3 and executes a variety of image processings; a memory module 12 in which the air-image and the subject image, the absorption rate and the variation rate, and add-value and the distance function, which will be described later, are written and memorized; a data and command input module 13; a display module 14 that displays the image that is obtained by the image generation/processing module 11; and a controller 15 that controls comprehensively these modules. Other than these, such as a high voltage generation module generates a high voltage and provides the tube electric current and the tube voltage to the X-ray tube 2 is also included but it is not shown in FIGs. because the present invention is not characterized by such modules and related modules thereof.

The memory module 12 writes and memorizes the data obtained by the image generation/processing module 11 through the controller 15 and reads out and send the data as needed from time to time to the display module 14 through the controller 15. The memory module 12 consists of memory media typically including a ROM (read-only memory), a RAM (random-access memory) and a hard-disk. Particularly, the memory module 12 of the present Embodiment includes the first absorption rate memory area 12a (referring to FIG. 4) and the second image memory area 12b (referring to FIG. 4).

The input module 13 sends the data and the command input by an operator to the controller 15. The input module 13 consists of a pointing device typically including a mouse, a key board, a joy stick, a trackball and a touch panel. The display module consists of a monitor.

The above image generation/processing module 11 and the controller 15 consist of a central processing unit (CPU) and so forth. The data obtained by the image generation/processing module 11 are written and memorized in the memory module 12 through the controller 15 or sent to and displayed in the display 14. The specific composition of the image generation/processing module 11 will be set forth later in detail.

Referring to FIG. 2, the FPD 3 consists of a plurality of X-ray detection elements d sensitive to X-ray, which are arrayed as two dimensional matrix (in the lengthwise and breadthwise) on the detection surface thereof. The X-ray detection element d once converts the X-ray transmitted the subject M to the X-ray detection signal (electric signal) and accumulates, and then detects the X-ray by reading out the accumulated X-ray detection signals. The X-ray detection signal detected by the respective X-ray detection elements d is converted to the pixel value corresponding to the X-ray detection signals, and the image generation module 21 (referring to FIG. 4) of the image generation/processing module 11 generates the X-ray image by assigning the pixel value to the pixel thereof respectively corresponding to the position of the X-ray detection element d.

Referring to FIG. 3, the grid 4 consists of the grid foil 4a that absorbs the scatter X-ray and the interlayer 4b transmitting X-ray, which are alternatively installed side-by-side. A grid cover 4c covering the grid foil 4a and the interlayer 4b sandwiches the grid foil 4a and the interlayer 4b from the incident face of X-ray and the opposite side face thereof. The grid cover 4c is illustrated as a dashed-two dotted line to clarify the grid foil 4a in the figure, and other components of the grid 4 (e.g., a mechanism supporting the grid foil 4a) are not shown in FIG. The grid foil 4a is corresponding to the grid foil of the present invention.

Further, referring to FIG. 3, the grid 4 is in place as each grid foil 4a is in place parallel to the detection face of the FPD 3. Further, the interlayer 4b of the present Embodiment is an empty gap and the grid 4 is also an air-grid. The grid foil 4a may be made of lead but not particularly limited to as long as the material is capable of absorbing a radiation, typically X-ray. Further, according to the present Embodiment, despite the focused grid in which the grid 4a is in place along the radiation line connecting the focal points of the X-ray tube 2 (referring to FIG. 1), each grid foil 4a is in place parallel for the convenient sake in FIG. 3.

Referring to FIG. 3, if each pixel size is AX, the grid foil 4a is in place as the foil shade can be projected in synchronism with each pixel. The grid foil 4a is in place as the foil shade can be projected in synchronism with the plural (e.g., 4) pixels. Accordingly, the grid foil 4a absorbs the X-ray so that the foil shade takes place in the FPD 3 and the foil shade is projected in the X-ray image, but the grid foil 4a is in place as the foil shade is projected in synchronism with respective pixels. Accordingly, the grid 4 is a synchronous type grid, wherein the grid foil 4a is in place as the foil shade is projected in the integral multiplicity (e.g., 4 times) of the distance (pixel pitch) between the pixels.

Referring to FIG. 3, the grid foil 4a is arrayed parallel in the x-direction (line direction) so that the grid foil 4a extends in the y-direction (column direction). Further, the X-ray detection elements d line up lengthwise and breadthwise and parallel in the x and y direction so that the grid foils 4a can be in place parallel in the x-direction of the X-ray detection elements d.

Next, the inventor illustrates the image generation/processing module and a series of the image processing flows, referring to FIG. 4-FIGS. 7A, 7B. FIG. 4 is a block diagram illustrating specific image generation/processing module of Embodiment, FIG. 5 is a flow diagram of a series of image processing, FIGS. 6A, 6B are a schematic view illustrating one Embodiment of the peak pixel coordinate/positional relationship of foil shade coordinate, the CP-value (direct X-ray transmission rate) and an amount of foil shade, and FIGS. 7A, 7B are related schematic views illustrating a variation of the grid foil shade and the profile of the direct X-ray transmission rate due to the position shift of the FPD/the grid.

Referring to FIG. 4, the image generation/processing module 11 comprises the image generation module 21, the first absorption rate calculation module 22, the second absorption rate calculation module 23, the variation rate calculation module 24, the add-value calculation module 25, the distance function calculation module 26, the selection module 27 and the image processing module 28. Further, the memory module 12 includes the first absorption rate memory area 12a and the second image memory area 12b. The image generation module 21 corresponds to the image generation means of the present invention, the first absorption rate calculation module 22 corresponds to the first absorption rate calculation means of the present invention, the second absorption rate calculation module 23 corresponds to the second absorption rate calculation means of the present invention, the variation rate calculation module 24 corresponds to the variation rate calculation means of the present invention, the add-value calculation module 25 corresponds to the add-value calculation means of the present invention, the distance function calculation module 26 corresponds to the distance function calculation means of the present invention, the selection module 27 corresponds to the selection means of the present invention, the image processing module 28 corresponds to the image processing means of the present invention and the first absorption rate memory area 12a corresponds to the first absorption rate memory means of the present invention.

Here, the inventor defines the terms used to set forth the present Embodiment of the present specification. The peak pixel is the pixel least affected by the foil shade of the grid foil 4a (referring to FIG. 3), which is in the intermediate position between the foil shade and the adjacent foil shade and is the pixel as far as possible from the foil shade. The x-coordinate of the peak pixel is the peak pixel coordinate. In contrast, the foil shade pixel is the pixel most affected by the foil shade of the grid foil 4a, which pixel is on the foil shade and is the pixel as close as possible to the foil shade. The x-coordinate of the foil shade pixel is the foil shade coordinate. As illustrated above, if the direct X-ray amount in case of no grid is “1.0”, the CP-value is the direct X-ray transmission coefficient in one pixel. Further, if the direct X-ray absorption rate of the grid foil 4a in one pixel is the foil shade amount, the value of the pixel having no grid shade should be “0.0” and the value of the pixel having the grid foil shade is “1.0—CP-value.” Further, when one peak pixel coordinate in one line of the pixel lines is remarked, the sum of the foil shade amounts relative to the pixel between remarkable peak pixel coordinates is the foil shade integration value. However, the peak pixel is the pixel as far as possible from the foil shade so that the foil shade integration values may not include the foil shade value on the peak pixels, given the foil shade cannot be projected on the peak pixels regardless such as distortion.

Further, the inventor defines the letters used to set forth the present Embodiment of the present specification. An x-coordinate of a pixel is x, a y-coordinate of a pixel is y, a grid foil 4a is number g, a peak pixel in one line is number p, an X-ray image (air-image) obtained under a predetermined condition without a subject is number a, the number of entire pixels (total pixel number) of the breadthwise size (x-direction size) of the shooting image is X_{SIZE}, the number of entire pixels (total pixel number) of the lengthwise size (y-direction size) of the shooting image is Y_{SIZE}, the total number of the grid foils 4a is N_{G}, the total number of the peak pixel lines is Nₚ (however, Nₚ=N_{G}+1) and the total number of the air-images is N_{A}. Then, 0≦x<X_{SIZE}, 0≦y<Y_{SIZE}, 0≦g<N_{G}, 0≦p<N_{P}, and 0≦a<N_{A} are obtained.

As illustrated above, according to the present Embodiment, N_{A} is a pattern number of position shifts of the two focal points of the X-ray tube relative to the FPD 3. At this time, the air-image are respectively taken at the pattern number of the position shifts of two focal points of the X-ray tube relative to the FPD every respective SID while changing SID every Zf (referring to FIG. 1) to obtain the total number N _{A} of the air-images.

Further, A (x, y, a) is the pixel value of air-image (hereafter simply “air-image”) at the number a, I (x, y) is the pixel value of the subject image (hereafter simply “subject image”), A_CP (x, y, a) is the image (hereafter simply the CP-value image) in which the CP-values calculated from the air-image are assigned to the pixels and lined up, and I_CP (x, y, a) is the CP-value image calculated from the subject image.

Referring to FIG. 4, the image generation module 21 generates the X-ray image based on the X-ray detection signals detected by the FPD 3 (referring to FIG. 1-FIG. 3.) Specifically, the image generation module 21 respectively generates a plurality of the X-ray images without a subject (specifically, air-image A (x, y, a)) based on the X-ray detection signals detected without the subject, i.e., setting no subject between the X-ray tube 2 (referring to FIG. 1) and the FPD 3. On the other hand, the image generation module 21 generates the X-ray image of the subject M (specifically, subject image I (x, y)) based on the X-ray detection signals detected with the subject M, i.e., setting the subject M between the X-ray tube 2 (referring to FIG. 1) and the FPD 3.

Specifically, the image generation module 21 respectively generates a plurality of the air-images A (x, y, a) as well as the subject image I (x, y). The subject image I (x, y) is written and stored in the second image memory area 12b of the memory module 12 through the controller 15 (not shown in FIG. 4.) Before taking the subject image, a plurality of air-images A (x, y, a) are respectively taken by the air-image shooting and then the subject images are taken by the subject shooting to generate the subject image I (x, y) and store in the second image memory area 12b.

The first absorption rate calculation module 22 calculates the direct X-ray absorption rate of each grid foil 4a relative to the air-image A (x, y, a.) Specifically, as illustrated above, the direct X-ray absorption rate of each grid foil 4a relative to the air-image A (x, y, a). the value of the pixel having grid shade should be “0.0” and the value of the pixel having no grid shade should be “1.0—CP-value” so that the first absorption rate calculation module 22 can obtain the direct X-ray absorption rate based on the CP-value image A_CP (x, y, a) calculated from the air-image. The CP-value image A_CP (x, y, a) calculated from the air-image is represented by the following formula (1).

A_CP(x, y, a)=A(x, y, a)/Spline {A(x, y, a)} Formula (1)

Here, Spline {A (x, y, a)} is a spline interpolation of the air-images (x, y, a) in the x-axis direction. However, as no scatter X-ray takes place because of no subject according the above Formula 1, the air-images do not include components of the scatter X-ray S {I (x, y), A (x, y, a)}, which is different from Formula (4) and Formula (5) illustrated later.

(Step S1) A_SHsum (g, y, a) is calculated.

Further, as illustrated in Step 1 of a flow diagram of FIG. 5, the first absorption rate calculation module 22 calculates the sum of each grid foil 4a absorption rate every pixel unit in-between thereof as one unit from a least affected pixel to another least affected pixel (i.e., between peak pixels) by the foil shade of each grid foil 4a among pixels of the plural air-images A (x, y, a). In the present specification, the sum of the absorption rates are respectively obtained every foil shade due to the grid foil 4a and assigned to the pixels to generate the images. The image as to the sum of the generated absorption rates is the foil shade integration value image calculated from the air-image and A-SHsum (g, y, a) is the foil shade integration value image calculated from the air-image. The foil shade integration value image A_SHsum (g, y, a) corresponds to the first absorption rate of the present invention.

Here, P_{A}(p, a) is the peak pixel coordinate the air-image at the number a. The peak pixel coordinates of the air-image are existing as the same number of air-images (in N_{A} ways) are represented as the function of two variables of p, a.

The foil shade integration value image A_SHsum (g, y, a) is calculated from the respective air-images A (x, y, a) using the following Formula (2).

$$\begin{matrix} Mathematical Formula 1 & \\ A\_SHsum ( g , y , a ) = w - {\sum }_{i = 1}^{w} A\_CP \{ {P}_{A} ( g , a ) + i , y , a \} & Formula ( 2 ) \\ w = {P}_{A} ( g + 1 , a ) - {P}_{A} ( g , a ) - 1 & Formula ( 3 )\end{matrix}$$

Here, w in Formula (3) is the pixel number between the foil shade pixel in one line of the pixel lines and the adjacent foil shade pixel thereto, in which the peak pixel is included in-between the foil shade pixel and the adjacent foil shade pixel.

The CP-value image A_CP (g, y, a) calculated from the air-images is also the direct X-ray transmittance rate, in which the maximum value of the direct X-ray amount (i.e., the direct X-ray amount in case of no foil shade) is also a normalized parameter to “1.0.” Accordingly, the pixel number w that is the first term of the right-hand side of Formula (2) becomes the parameter in which the total radiation amount between the foil shade pixel in one line of the pixel lines and the adjacent foil shade pixels are normalized, and one pixel without foil shade coincides with the maximum value “1.0” of the direct X-ray amount. On the other hand, as the second term of the right-hand side of Formula (2) is the sum of the CP-value image A_CP (g, y, a) relative to the pixel between the foil shade pixel in one line of pixel lines and the adjacent foil shade pixel thereto, the second term is subtracted from the first term in the right-hand side of the above Formula (2) so that the foil shade integration value image A-SHsum (g, y, a) that is normalized to the sum of the absorption rate of each grid foil 4a every interval between pixel units can be obtained.

Accordingly, the first absorption rate calculation module 22 calculates the direct X-ray absorption rate of each grid foil 4a relative to the air-image A (x, y, a) and the foil shade integration value image A-SHsum (g, y, a) that is normalized to the sum of the absorption rate of each grid foil 4a every interval between pixel units. The foil shade integration value image A-SHsum (g, y, a) is written and stored in the first absorption rate memory area 12a of the memory module 12 through the controller 15 (not shown in FIG. 4.)

Meantime, the air-image is taken just before the subject shooting in each case, and the foil shade integration value image A-SHsum (g, y, a) is calculated in each case but a variety of operations later set forth are not required using the foil shade integration value image A-SHsum (g, y, a) in each case. It is preferable that the foil shade integration value image A_SHsum (g, y, a) is calculated to be written into the first absorption rate memory area 12a and stored based on the advanced air-image shooting, and then a variety of operations set forth later are conducted by commonly using the foil shade integration image A_SHsum (g, y, a) stored in the first absorption rate memory area 12a. Specifically, referring to FIG. 4, the foil shade integration value image A_SHsum (g, y, a) is calculated in advance with the first absorption rate memory area 12a and is written to store in the first absorption rate memory area 12a so that numbers of a variety of operations can be reduced.

The foil shade integration value image A_SHsum (g, y, a) calculated in the first absorption rate calculation module 22 and stored in the first absorption rate memory area is read out from the first absorption rate memory area 12a and sent to the second absorption rate calculation module 23, the variation rate calculation module 24 and the selection module 27.

On the other hand, the second absorption rate calculation module 23 calculates the direct X-ray absorption rate of each grid foil 4a relative to the subject image I (x, y) by reading out the subject image (x, y) stored in the second image memory area (12b). Specifically, as illustrated above, the direct X-ray absorption rate of each grid foil 4a relative to the subject image (x, y) the value of the pixel having grid shade should be “0.0” and the value of the pixel having no grid shade should be “1.0—CP-value” so that the first absorption rate calculation module 23 can obtain the direct X-ray absorption rate based on the CP-value image I_CP (x, y, a) calculated from the subject image. The CP-value image I_CP (x, y, a) calculated from the subject image is represented by the following Formula (4).

Mathematical Formula

I_CP(x, y, a)=G(x, y, a)/Spline {G(x, y, a)} Formula (4)

G(x, y, a)=I(x, y)−S{I(x, y), A(x, y, a)} Formula (5)

Here, as the scatter X-ray takes place because of the intervened subject M according to the above formula (4) and formula (5), the subject images include components of the scatter X-ray S {I (x, y) , A (x, y, a)}. Therefore, according to Formula (5), the image in which the component of the scatter X-ray S {I (x, y), A (x, y, a)} is removed from the subject image I (x, y) is G (x, y, a). Spline {G (x, y, a)} is a spline interpolation of G (x, y, a) in the x-axis direction. Further, the scatter X-ray S {I (x, y), A (x, y, a)} can be obtained from the above illustrated foil shade integration value image A_SHsum (g, y, a).

Accordingly, the second absorption rate calculation module 23 reads out not only the subject image I (x, y) stored in the second image memory area 12b but also the foil shade integration value image A_SHsum (g, y, a), and calculates the scatter X-ray S {I (x, y), A (x, y, a)} based on the subject image I (x, y) and the foil shade integration value image A-SHsum (g, y, a). Then, the direct X-ray absorption rate of each grid foil 4a relative to the subject image I (x, y) using the scatter X-ray S {(x, y) , A (x, y, a)} is calculated. Accordingly, the second absorption rate calculation module 23 reads out and employs the first absorption rate (foil shade integration value image A_SHsum (g, y, a)) to conduct the operation to calculate the second absorption rate (foil shade integration value image I_SHsum (g, y, a).

(Step S2) I_SHsum (g, y, a) is calculated.

Further, as illustrated as Step 2 in the flow diagram FIG. 5, the second absorption rate calculation module 23 calculates the sum of the absorption rate of each grid foil 4a every pixel unit in-between thereof as one unit from a least affected pixel to another least affected pixel (i.e., between peak pixels) by the foil shade of each grid foil 4a among pixels of the subject image I (x, y). In the present specification, the sum of the absorption rates are respectively obtained every foil shade due to the grid foil 4a and assigned to the pixels to generate the images. The image as to the sum of the generated absorption rates is the foil shade integration value image calculated from the subject image and I-SHsum (g, y, a) is the foil shade integration value image calculated from the subject image. The foil shade integration value image I_SHsum (g, y, a) corresponds to the second absorption rate of the present invention.

Herein, P₁ (P) is the peak pixel coordinate of the subject image. The subject image is the data including only one subject image, which is different from the peak pixel coordinate of the air-image, so that it can represent the peak pixel coordinate of the subject as one variable function relative to only p.

The foil shade integration image I-SHsum (g, y, a) is calculated from the subject image I (x, y) using the following formula (6.)

$$\begin{matrix}Mathematical Formula 2 & \\ I\_SHsum ( g , y , a ) = w - {\sum }_{i = 1}^{w} I\_CP \{ {P}_{I} ( g ) + i , y , a \} & Formula ( 6 ) \\ w = {P}_{I} ( g + 1 ) - {P}_{I} ( g ) - 1 & Formula ( 7 )\end{matrix}$$

Here, w in Formula (7), as illustrated as to w in the above formula (3), is the pixel number between the foil shade pixel in one line of the pixel lines and the adjacent foil shade pixel thereto, in which the peak pixel is included in-between the foil shade pixel and the adjacent foil shade pixel.

As illustrated as to the air-image, the CP-value image I_SHsum (g, y, a) calculated from the subject images is also the direct X-ray transmittance rate, in which the maximum value of the direct X-ray amount (i.e., the direct X-ray amount in case of no foil shade) is a normalized parameter to “1.0”. Accordingly, the pixel number w that is the first term of the right-hand side of Formula (6) becomes the parameter in which the total radiation amount between the foil shade pixel in one line of the pixel lines and the adjacent foil shade pixels is normalized, and one pixel without foil shade coincides with the maximum value “1.0” of the direct X-ray amount of one pixel. On the other hand, as the second term of the right-hand side of Formula (6) is the sum of the CP-value image I_SHsum (g, y, a) relative to the pixel between the foil shade pixel in one line of pixel lines and the adjacent foil shade pixel thereto, the second term is subtracted from the first term in the right-hand side of the above formula (6) so that the foil shade integration value image I-SHsum (g, y, a) that is normalized to the sum of the absorption rate of each grid foil 4a every interval between pixel units can be obtained.

Accordingly, the second absorption rate calculation module 23 calculates the direct X-ray absorption rate of each grid foil 4a relative to the subject image I (x, y, a) and I-SHsum (g, y, a) that is normalized to the sum of the absorption rate of each grid foil 4a every interval between pixel units. The foil shade integration value image I_SHsum (g, y, a) calculated by the second absorption rate calculation module 23 is sent to the variable calculation module 24. Practically, the foil shade integration value image I_SHsum (g, y, a) calculated by the second absorption rate calculation module 23 is written and stored in the memory 12 through the controller 15 (not illustrated in FIG. 4) and when the variation rate is calculated by the variation calculation module 24 as later set forth, the foil shade integration value image I-SHsum (g, y, a) stored in the memory 12 is read out and employed.

The peak pixel coordinate of the air-image P_{A} (p. a) is p [a] in FIG. 6B, p [a+1], and pₙ, pₙ₊₁ in FIGS. 7A, 7B. As illustrated in FIG. 6A, when the total number of the grid foil 4a (referring to FIG. 3) is N_{G} and if the number is assigned as “0”, “1”, “2” from the left end, the right end is N_{G}−1. The middle of FIG. 6B is the expansion of the pixel lines of the air-image in FIG. 6B. As illustrated in the middle of FIG. 6B, the pixel coordinates are numbered as “0”, “1”, “2” in order from the left side of the pixel coordinate; and the foil shade coordinate of the foil number a (a) is g [a] ; the peak pixel coordinate between the foil shade coordinate g [a−1] of the foil number (a−1) and the foil shade coordinate g [a] of the foil number a is p [a] and the peak pixel coordinate between the foil shade coordinate g [a] of the foil number a and the foil shade coordinate g [a+1] of the foil number (a+1) is p [a+1] . The lower side of FIG. 6B is the expansion of the foil number “N_{X}−1”of the middle of FIG. 6B.

As illustrated in the lower side of FIG. 6B, given the pixel width is t, the X-ray transmission widths are u and v, the CP-value is CP-value=(u+v)/t and the foil shade amount is Foil shade amount={t−(u+v)}=1.0−CP-value. Further, referring to FIGS. 6A, 6B, for the convenience for sake, the aspect in which the foil shading having the width of {t−(u+v)} is projected, but the pixel having the minimum pixel width t constitutes the pixel so that it should be noticed that the foil shade narrower than the pixel width t is not projected in the actual pixel and projected evenly to the foil shade pixel corresponding to 1 pixel that is the pixel value in which X-ray is absorbed by the grid foil.

Further, FIG. 7A illustrates the profile without the position shift of the FPD/grid and the lower part in FIG. 7A illustrates the expansion profile of the grid foil shade and the direct X-ray transmission rate without the position shift of the FPD/grid, and FIG. 7B illustrates the profile with the position shift of the FPD/grid and the lower part in FIG. 7B illustrates the expansion profile of the grid foil shade and the direct X-ray transmission rate with position shift. Referring to the lower part of 7A and the lower part of FIG. 7B, the pixel coordinate between the peak pixel coordinate pₙ and the peak pixel coordinate pₙ₊₁ are a, b, c in order from left. Referring to FIGS. 7A, 7B, the foil shades are projected in synchronism with 4 pixels.

At this time, when no position shift of the FPD/grid takes place, the grid foil shade is projected only in the pixel corresponding to the pixel coordinate b at the middle of the peak pixel coordinate pₙ and peak pixel coordinate pₙ₊₁, and the profile of the direct X-ray transmission rate plunges at only the pixel corresponding to the pixel coordinate b, but is “1.0” at other pixel corresponding to the pixel coordinate a and the pixel coordinate c than that pixel. On the other hand, if the grid foil shade is projected bridging over both the pixel coordinate b and the pixel coordinate c between the peak pixel coordinate pₙ and the peak pixel coordinate pₙ₊₁ when the position shift of the FPD/grid takes place, the profile of the direct X-ray transmission rate will plunge at pixels' position corresponding to the pixel coordinate b and the pixel coordinate c but will remain as “1.0” at pixels' position corresponding to the pixel coordinate a.

Accordingly, as set forth in “Problem to be Solved”, if the position shift of the gird relative to the FPD takes place, the profile of the grid foil shade would change as illustrated in FIGS. 7A, 7B. In contrast, as to the peak pixels in-between (pₙ, pₙ₊₁) that are least impacted by the foil shade due to the grid foil, it is understandable that that the sum (referring the area of the lower gray part in FIGS. 7A, 7B) of the absorption direct X-ray amount (foil shade amount) due to the grid foil will not change even if the position of the grid relative to the FPD shifts. Specifically, when no position shift of the FPD/grid takes place, the sum of the foil shade at the lower part in FIG. 7A (i.e., the sum of the absorption rates) is the triangle area surrounded by a, b, and c, but if the position shift of the FPD/grid takes place, the sum of the foil shade at the lower part in FIG. 7B (i.e., the sum of the absorption rates) is the quadrangular area surrounded by a, b, c, p₊₁ so that both areas do not change.

According to the above rationale, the correlation is obtained by calculating the distance function, later set forth, is calculated by calculation of the sum of the absorption rates of each grid foil every in-between of unit pixels thereof as one unit from a least affected pixel to another least affected pixel (between peak pixels) due to the foil shade relative to each grid foil among pixels of each image so that the limitation as to the positional relationship between the FPD and the grid which must be the same both times on the air-image shooting and on the subject shooting can be alleviated. According to the present Embodiment, as illustrated with regards to the Step S1, S2 of the flow diagram in FIG. 5, the foil shade integration value image A_SHsum (g, y, a) and the foil shade integration image I_SHsum (g, y, a), which are normalized to the sum of the absorption rates of each grid foil 4 every in-between of unit pixels, are respectively calculated.

The inventor returns to illustration of FIG. 4 and FIG. 5.

Referring to FIG. 4, the variation rate calculation module 24 calculates the variation rate of the subject image relative to the air-image based on the direct X-ray absorption rate of each grid foil 4a (referring to FIG. 3) relative to the plural air-images A (x, y, a) which is calculated by the first absorption rate calculation module 22 and stored in the first absorption rate memory area and read out therefrom and the direct X-ray absorption rate of the each grid foil 4a relative to the subject images I (x, y) calculated by the second absorption rate calculation module 23.

(Step S3) R (g, y, a) is calculated.

According to the present Embodiment, as illustrated with regards to the Step S3 of the flow diagram in FIG. 5, the variation rate is calculated based on the number N_{A} of the foil shade integration value image A_SHsum (g, y, a) and the foil shade integration value image I_SHsum (g, y, a) calculated from the subject images. Given R (g, y, a) is the variation rate, the variation rate R (g, y, a) is calculated using the following formula (8) from the foil shade integration value image A_SHsum (g, y, a) and the foil shade integration value image I_SHsum (g, y, a), which are normalized to the sum of the absorption rates of each grid foil 4a.

R(g, y, a)=I_SHsum (g, y, a)/A_SHsum (g, y, a) Formula (8)

According to Formula (8), the variation rate is obtained by dividing the foil shade integration value image I_SHsum (g, y, a) calculated from the subject image by the foil shade integration value image I_SHsum (g, y, a) calculated from each air-image.

Accordingly, the variation calculation module 24 calculates the variation rate R (g, y, a) of the subject image relative to the air-image. The variation rate R (g, y, a) calculated by the variation rate calculation module 24 is sent to the add-value calculation module 25. Practically, the variation R (g, y, a) calculated by the variation calculation module 24 is written and stored in the memory 12 through the controller 15 (not sown in FIG. 4) and when the add-value is calculated by the add-value calculation module 25, as later set forth, the variation R (g, y, a) stored in the memory module 12 is read out and employed. Accordingly, the variation calculation module 24 reads out and employs the first absorption rate (foil shade integration value image A_SHsum (g, y, a), which is stored in the first absorption rate memory area 12, to conduct the operation to calculate the variation R (g, y, a).

The add-value calculation module 25 adds the variation R (g, y, a) calculated by the variation calculation module 24 along the direction (column direction, y-direction, in FIG. 3 of the present Embodiment) in which the grid foil 4a is extending or executes the arithmetic average (add-average) that is added and then averaged And the add-value is calculated using such add or such arithmetic average (add-average.) According to the present Embodiment, the add-average process relative to the variation R (g, y, a) of the up-and-down 64 pixels corresponding to one line (total 129 pixels including pixels corresponding to the central pixel coordinate “0”) as the central pixel coordinate “0” relative to y-direction is executed and the average variation of the CP integration values is calculated, If AveR (g, a) is the average variation, the average variation AverR (g, a) is calculated using the following Formula (9.)

$$\begin{matrix}Mathematical Formula & \\ AveR ( g , a ) = \frac{1}{129} {\sum }_{i = - 64}^{64} R ( g , t arg ety + i , a ) & Formula ( 9 )\end{matrix}$$

Here, the target y of Formula (9) is the target y-coordinate and the central pixel coordinate is “0”. Further, the target y-coordinate is not limited to the central pixel coordinate “0”, and the target y-coordinate may be the lower end pixel coordinate or the target y-coordinate may be the upper end pixel coordinate. Further, the average variation AveR (g, a) is not limited to the 1 line add-value (add-average value), and the add-average range may include a few upper or lower pixels from the target y-coordinate as the center excluding upper and lower ends. Further, the add-average range may include the upper and lower 64 pixels but the specific range of the add-average range is not particularly limited.

The upper and lower add-average processing may have an effect on decreasing the impact due to the noise of the image when the distance function, descried later, is calculated. Accordingly, if the add-average range is adjusted to the noise level and the calculation range of the foil shade integration value, the CP-value, and the CP-integration value is limited according to the add-average range, the processing rate can be expected higher.

Accordingly, the add-value calculation module 25 calculates the average variation AveR (g, a) as the add-value using such add or such arithmetic average (add-average.) The average variation rate R (g, a) calculated by the add-value calculation module 25 is sent to the distance function calculation module 26. Practically, the average variation rate AveR (g, a) calculated by the add-value calculation module 25 is written and stored in the memory 12 through the controller 15 (not illustrated in FIG. 4) and when the distance function is calculated by the distance function calculation module 26, as later set forth, the average variation rate AveR (g, a) stored in the memory 12 is read out and employed.

The distance function calculation module 26 calculates the distance function based on the average variation rate AveR (g, a) calculated by the add-value calculation module 25. The average variation rate AveR (g, a) is ideally “1.0”. Then, the distance from the ideal value is calculated every grid foil 4a, and if the average value is the distance D and the distance function D (c), the distance function D (c) is calculated using the following Formula (10).

$$\begin{matrix}Mathematical Formula & \\ D ( c ) = \frac{I}{{\sum }_{i = 0}^{{N}_{G} - 1} 1} {\sum }_{i = 0}^{{N}_{G} - 1} \vert 1.0 - AveR ( i , c ) \vert & Formula ( 10 )\end{matrix}$$

Here, the denomination of the above Formula (9) is the total number N_{G} of the grid foil 4a, and the average value every foil is obtained by dividing the total number N_{G} of the grid foil 4a.

Accordingly, the distance function calculation module 26 indicates the correlation level between the air-image and the subject image and also calculates the distance function D (c) indicating the distance of the grid foil 4a from the ideal value. The distance function D (c) calculated by the distance function calculation module 26 is sent to the selection module 27. Practically, the distance function D (c) calculated by the distance function calculation module 26 is written and stored in the memory 12 through the controller 15 (not illustrated in FIG. 4) and when the first absorption rate is selected by the selection module 27, as later set forth, the distance function D (c) stored in the memory 12 is read out and employed.

(Step S4) The minimum value of the distance function D (c) is searched.

Referring to Step 4 of the flow diagram in FIG. 5, the foil shade integration value image A_SHsum (g, y, a) stored in the first absorption memory 12a is read out and the selection module 27 selects the first absorption rate (foil shade integration value image A_SHsum (g, y, a), at which the distance function D (c) calculated by the distance function calculation module 26 becomes minimum, from the plural foil shade integration value images A_SHsum (g, y, a.) Specifically, the range of the distance is 0≦D and the larger correlation of the CP-integration value between the air-image and the subject image is obtained, the closer the distance D is to “0.” Accordingly, the first absorption rate providing the minimum distance function D (c) is selected as the optimum first absorption rate. Referring to the flow diagram in FIG. 5, when the selection takes place, it is optimum (but d 0≦optimum<N_{A}) and the selected first absorption rate is A_SHsum (g, y, optimum).

The section module 27 selects the first absorption rate as the optimum integration value image A_SHsum (g, y, optimum), at which the distance function D (c) calculated by the distance function calculation module 26 becomes minimum, from the plural foil shade integration value images A_SHsum (g, y, a.) The foil shade integration value image A_SHsum (g, y, optimum) selected by the selection module 27 is sent to the image processing module 28. Practically, the foil shade integration value image A_SHsum (g, y, a) selected by the selection module 27 is written and stored in the memory 12 through the controller 15 (not illustrated in FIG. 4) and when the image processing is executed by the image processing module 28, as later set forth, the foil shade integration value image A_SHsum (g, y, optimum) stored in the memory 12 is read out and employed. Further, the foil shade integration value image A_SHsum (g, y, optimum) may be written and stored in the first absorption memory area 12a, but it is preferable that the selected foil shade integration value image A_SHsum (g, y, optimum) is written and stored in the different area to distinguish from the foil shade integration value image A_SHsum (g, y, a) before selection. Accordingly, the selection module 27 reads out and employs the first absorption rate (foil shade integration value image A_SHsum (g, y, a) to conduct the operation to select the first absorption rate as the optimum foil shade integration value image A_SHsum (g, y, a) at which the distance function D (c) becomes minimum.

The foil shade integration value image A_SHsum (g, y, optimum) that is selected by the selection module 27 and stored in the memory module 12 is read out and also the subject image I (x, y) stored in the second image memory area 12b is read out. Then, an image processing module 28 that executes the image processing to obtain the X-ray image by removing the foil shade due to the grid foil 4a of the subject image I (x, y) and calculating the direct X-ray component of the subject M based on the foil shade integration value image A_SHsum (g, y, optimum) selected by the selection module 27 and the subject image I (x, y).

The method for the image processing to obtain the X-ray image of which the foil shade is removed based on the foil shade integration value image A_SHsum (g, y, optimum) and the subject image I (x, y) is not particularly limited. For example, the optimized CP-value is calculated based on the foil shade integration value image A_SHsum (g, y, optimum) and then the X-ray image of which the foil shade is removed may be obtained by dividing the optimized CP-value from the subject image I (x, y). In this case, as the first absorption rate is the optimal foil shade value image A_SHsum (g, y, optimum) is selected to provide the minimum distance function D (c), each foil shade pixel of the subject image I (x, y) would be respectively corresponding to each foil shade pixel of the foil shade integration value image A_SHsum (g, y, optimum) so that an effect on that a simple division of the pixel value of only foil shade pixel can easily remove the foil shade can be provided.

Accordingly, the image processing module 28 writes and stores the X-ray image, of which the foil shade is removed, in the memory module 12 through the controller 15 (not shown in FIG. 4.) If needed, the X-ray image stored in the memory module 12 may be read out and displayed on the display module 14 (referring to FIG. 1) and/or printed out by a printing means represented by a printer.

According to the X-ray device of the present Embodiment, the first absorption rate calculation module 22 calculates the absorption rate of the direct X-ray of each grid foil 4a, as the first absorption rate, relative to a plurality of X-ray images (air-images) without a subject based on the X-ray detection signals detected without the subject that would be the subject set between the X-ray tube 2 and the FPD 3. On the other hand, the second absorption rate calculation module 23 calculates the absorption rate of the direct X-ray of each grid foil 4a, as the second absorption rate, relative to X-ray images with the subject M based on the X-ray detection signals detected with the subject M that would be the subject set between the X-ray tube 2 and the FPD 3.

Then, the distance function calculation module 26 calculates the distance function based on the first absorption rate calculated by the first absorption rate calculation module 22 and the second absorption rate calculated by the second absorption rate calculation module 23. As described above, the distance function indicates the correlation level between the air-image and the subject image and also is the parameter indicating the distance of the grid foil 4a from the ideal value.

The selection module 27 selects the first absorption rate from a plurality of the first absorption rates, wherein the distance function calculated by the distance function calculation module 26 becomes minimum. Specifically, the larger correlation between the air-image and the subject image, the closer the distance to the ideal value and the distance function closes to “0”. Accordingly, the first absorption rate providing the minimum distance function is selected as the optimum absorption rate. Then, an image processing module 28 executes the image processing to obtain the x-ray image by removing the foil shade due to the grid foil 4a of the x-ray image of the subject and calculating the component of the direct x-ray of the subject M based on the first absorption rate and the subject image selected by the selection module 27.

Accordingly, given a plurality of the first absorption rates based on the distance function are selected, the scatter x-ray and the grid foil 4a can be removed without preparing a number of patterns despite the position shift of the grid 4 relative to the FPD 3. Further, according to the present Embodiment, only a number N_{A} of the air-image can be taken in advance so that the shooting number of the air-image can be decreased and also an effective on that the memory area to keep the air-images can be decreased can be provided.

According to the present Embodiment, the first absorption rate calculation module 22, the second absorption rate calculation module and the distance function calculation module 26 calculate specifically as described above. Specifically, the first absorption rate calculation module 22 calculates the direct X-ray absorption rate of each grid foil 4a relative to each pixel of the plural air-images and the sum (foil shade integration value images A_SHsum (g, y, a) normalized to the sum of the absorption rate) of the absorption rate of each grid foil 4a absorption rate every pixel unit in-between thereof as one unit from a least affected pixel to another least affected pixel (i.e., between peak pixels) by the foil shade of each grid foil 4a among pixels of the plural air-images. The second absorption rate calculation module 22 calculates, almost as well as the first absorption rate calculation module 22, the direct X-ray absorption rate of each grid foil 4a relative to each pixel of the subject images and the sum of each grid foil 4a absorption rate every pixel unit in-between thereof as one unit from a least affected pixel to another least affected pixel (between peak pixels) by the foil shade of each grid foil 4a (according to the present Embodiment, I-SHsum (g, y, a) normalized to the sum of the absorption rate). The distance function calculation module 26 calculates the distance function based on the sum (foil shade integration value image A_SHsum (g, y, a)) of absorption rates of each grid foil 4a calculated by the first absorption rate calculation module 22 and the sum (foil shade integration value image I_SHsum (g, y, a) of second absorption rates of each grid foil 4a calculated by the second absorption rate calculation module 23.

According to the present Embodiment, it is preferable that a variation rate calculation module 24 is included. Specifically, the variation rate calculation module 24 calculates the variation rate of the subject image relative the air-image based on the direct X-ray absorption rate of each grid foil 4a relative to the plural air-images (according to the present Embodiment, foil shade integration value image A_SHsum (g, y, a) normalized to the sum of the absorption rate) and the direct X-ray absorption rate (according to the present Embodiment, foil shade integration value image I_SHsum (g, y, a) normalized to the sum of the absorption rate) of each grid foil 4a relative to the subject images calculated by the second absorption rate calculation module 23. The variation rate can be ideally “1”. The distance function calculation module 26 calculates the distance function based on the variation rate calculated by the variation rate calculation module 24 so that the larger correlation between the air-image and the subject image, the closer the variation rate to the ideal value “1” and the distance function closes to “0”. Accordingly, the distance function will be able to be calculated by using the variation rate.

Particularly, when the distance function is calculated by using the variation rate, according to the present Embodiment, it is useful when the grid 4 constituting the grid foil 4a arrayed parallel to only one direction (line direction of the present Embodiment) of either lengthwise or breadthwise direction of the X-ray detection element d as illustrated in FIG. 3 (referring to FIG. 2) is employed. The grid 4 having such structure, the grid foil 4a would not crossover in the grid foil 4a extending direction (when the grid foils 4a are arrayed parallel to the x-direction, the grid foils 4a extending direction is the column direction) so that the statistical error (i.e., noise) may take place in the extending direction. Then, in order to lower the impact due to the noise of the image, the variation rate calculated by the variation rate calculation module 24 along the extending direction of the grid foils 4a or conducts an additional averaging (so called “add-average”), which is adding and then averaging, to calculate the add-value due to the adding or additional averaging. According to the present Embodiment, the add-average processing relative to the pixel coordinate “0” of the up-and-down 64 pixels corresponding to one line (total 129 pixels including pixels corresponding to the central pixel coordinate “0”) is executed relative to the variation rate R (g, y, a) and the average variation rate AveR (g, a) is calculated. The distance function calculation module 25 calculates a distance function based on the add-value calculated by the add-value calculation module 25 (average variation rate AveR (g, a)) so that the impact due to the noise of the image may effectively decrease

According to the present Embodiment, referring to FIG. 4, it is preferable that the first absorption rate memory area 12a, in which the first absorption rate (foil shade integration value image A_SHsum (g, y, a) calculated by the first absorption rate calculation module 22 is written, is included and the first absorption rate (foil shade integration value image A_SHsum (g, y, a)) stored in the first absorption rate memory area 12a is read out and employed to execute a variety of operations (the second absorption rate calculation module 23, the variation calculation module 24 and the selection module 27). Given the air-image is written and memorized, every time when the first absorption rate (foil shade integration value image A_SHsum (g, y, a)) is calculated by the first absorption rate calculation module is operated, the first absorption rate (foil shade integration value image A_SHsum (g, y, a)) by reading out the air-image, but if the first absorption rate (foil shade integration value image A_SHsum (g, y, a)) calculated in advance is written and memorized, the number of operations can be effectively decreased. Further, the memory area, in which the air-image is written and memorized, requires a size for entire pixels (X_{SIZE}×Y_{SIZE}×N_{A}), but the size of the memory area, in which the first absorption rate (foil shade integration value image A_SHsum (g, y, a)) is written and memorized, decreases only for the grid foils so that the size of the memory area of the first absorption rate memory area 12a can be effectively decreased.

The present invention is not limited to the above Embodiment and further another alternative Embodiment can be implemented.

(1) In the above Embodiment, the radiation is X-ray but other radiation than X-ray (e.g., γ-ray) can be applied.

(2) In the above Embodiment, the X-ray shooting device is the C-arm X-ray fluoroscopic shooting device having and the C-arm to be implemented on the CVS device but not limited. For example, it can be the structure for the nondestructive inspection device employed in an industry, in which the subject (in this case, the inspection object is the subject) is being carried on the belt while shooting, and also can be the X-ray CT device in medicine.

(3) According to the above Embodiment, the air grid is adopted as the scatter radiation removal means, but not limited. Besides an air-gap, the grid can be an intermediate substance (spacer) which is made of e.g., aluminum and an organic substance through which the radiation represented by X-ray transmits. Further, the cross grid constituting the grid foils arrayed in both lengthwise and breadthwise directions (x, y directions) of the detection elements may be employed.

(4) According to the above Embodiment, the focus grid is applied, but it can be applied also for the scatter radiation removal means in which the grid foils are arrayed parallel.

(5) According to the above Embodiment, a synchronous type grid in which grid foils are in place as the foil shade is projected in the integral multiplicity of the distance (pixel pitch) between the pixels of the shooting image is illustrated, but it can be applied also to a non-synchronous type grid. Further, in the case of a grid other than the air-grid, the present invention can be applied to the grid having the structure in which a plurality of grid foils are installed side-by-side to one pixel. However, if a plurality of the grid foils are installed evenly across all over one pixel, all become foil shade pixel so that a peak pixel cannot be exist, and then the present invention cannot be applied. When a plurality of grid foils are installed side-by-side to one pixel, it is premised that a gird foil should not be installed over more than one pixel so that peak pixels can exist in proximity to grid foils.

(6) According to the above Embodiment, when the distance function is calculated based on the direct radiation absorption rate of each grid foil relative to the radiation image without a subject (air-image) and the direct radiation absorption rate of each grid foil relative to the radiation image of a subject (subject image), the variation rate is employed but the variation rate may not be employed. For example, each absorption rate is respectively normalized to obtain the difference value and then the distance function may be calculated using the difference value as a variable.

(7) According to the above Embodiment, the distance function is calculated using the grid 4, referring to FIG. 3, instead of a cross grid, based on the add-value that is added or add-averaged along the direction in which the grid foil is extending, but the invention is not limited to the present Embodiment. If the noise is small in the extending direction or in the case of using the cross grid, the variation rate R (g, y, a) before adding may be used to calculate the distance function.

It will also be understood, that as used herein the phrases module or means are linked with any necessary components to achieve the function; for example a calculation module that calculates will include an electronic memory, processing controller, recorded media that operates to calculate as directed and to take any and all other necessary steps to achieve the requirement identified; and further that those of skill in this related art will well understand how to prepare such ‘modules’ or other integrated devices and units as noted herein to function as noted or any portion thereof.

Having described at least one of the preferred embodiments of the present invention with reference to the accompanying drawings, it will be apparent to those skills that the invention is not limited to those precise embodiments, and that various modifications and variations can be made in the presently disclosed system without departing from the scope or spirit of the invention. Thus, it is intended that the present disclosure cover modifications and variations of this disclosure provided they come within the scope of the appended claims and their equivalents.

### EXPLANATION OF REFERENCES

2 - - - X-ray tube
3 - - - A flat panel type X-ray detector (FPD)

### 4 - - - Grid

4a - - - Grid foil
12a - - - First absorption rate memory area
21 - - - Image generation module
22 - - - First absorption rate calculation module
23 - - - Second absorption rate calculation module
24 - - - Variation rate calculation module
25 - - - Additional value calculation module
26 - - - Distance function calculation module
27 - - - Selection module
28 - - - Image processing module

### A (x. y. a) - - - Air-image

I (x, y) - - - Subject image
A_SHsum (g, y, a), I_SHsum (g, y, a) - - - Foil shade integration value image
R (g, y, a) - - - Variation rate
AveR (g, a) - - - Average variation rate

### M - - - Subject

What is claimed is:

1. A radiographic device, to obtain a radiation image, comprising:
a radiation source that irradiates a radiation;
a radiation detection module, wherein a plurality of radiation detection elements operative to detect the irradiated radiation are installed in a lengthwise and a breadthwise direction relative to each other;
a scatter radiation removal module, wherein a plurality of grid foils that operably absorb the scatter radiation are set in the detection side of the radiation detection module and installed side-by-side parallel in at least one of either the lengthwise or the breadthwise direction of the radiation detection elements;
a image generation module that generates the radiation image based on the radiation detection signal detected by said radiation detection module; and
said the radiographic device, further comprising:
a first absorption rate calculation module that calculates the absorption rate of the direct radiation of each grid foil as the first absorption rate relative to a plurality of radiation images without a subject based on the radiation detection signals detected without a subject that would be the subject set between said radiation source and said radiation detection module;
a second absorption rate calculation module that calculates the absorption rate of the direct radiation of each grid foil, as the second absorption rate, relative to radiation images with a subject based on the radiation detection signals detected with a subject that would be the subject set between said radiation source and said radiation detection means;
a distance function calculation module that calculates the distance function indicating the correlation level between the radiation image without said subject and the radiation image with said subject and also the distance of the grid foil from the ideal value, based on the first absorption rate calculated by the first absorption calculation module and the second absorption rate calculated by the second absorption calculation module;
a selection module that selects said first absorption rate from a plurality of the first absorption rates, wherein said distance function calculated by the distance function calculation module becomes a minimum; and
an image processing module that executes the image processing to obtain the radiation image in which said foil shade is removed by removing the foil shade due to the grid foil of the radiation image of the subject and calculating the component of the direct radiation of the subject based on said first absorption rate selected by the selection module and the radiation image of said subject.

2. The radiographic device, according to claim 1, where:
the first absorption rate calculation module calculates an absorption rate of the direct radiation of each grid foil relative to each pixel of the plural radiation images without said subject, calculates the sum of each grid foil absorption rate every pixel unit in-between thereof as one unit from a least affected pixel to another least affected pixel by the foil shade of each grid foil among pixels of the plural radiation images without the subject;
said second absorption rate calculation module calculates an absorption rate of the direct radiation of each grid foil relative to each pixel of the radiation images with said subject, calculates the sum of each grid foil absorption rate every pixel unit in-between thereof as one unit from a least affected pixel to another least affected pixel by the foil shade of each grid foil among pixels of the radiation images with said subject; and
said distance function calculation module calculates based on the sum of the first absorption rates calculated by the first absorption rate calculation module and the sum of the second absorption rates calculated by the second calculation module.

3. The radiographic device, according to claim 2, further comprising;
a variation rate calculation module that, calculates the variation rate of the radiation image of said subject relative to the radiation image without said subject, calculates based on the direct radiation absorption rate of each grid foil relative to the plural radiation images without said subject, calculated by said first absorption rate calculation module, and the direct radiation absorption rate of each grid foil relative to the radiation images with said subject, calculated by the second absorption rate calculation module;
wherein said distance function calculation module calculates said distance function based on said variation rate calculated by said variation calculation module.

4. The radiographic device, according to claim 3, wherein:
said scatter radiation removal module constitutes said grid foils arrayed parallel in either one direction of lengthwise or breadthwise of said radiation detection element;
the radiographic device, further comprising:
an add-value calculation module operative to calculate the add-value due to the adding or the additional averaging, wherein said variation rate calculated by said variation rate calculation module along the extending direction of the grid foils is added or averaged by an additional averaging;
the distance function calculation module calculates said distance function based on said add-value calculated by the add-value calculation module.

5. The radiographic device, according to claim 4, further comprising:
a first absorption rate memory module; and
wherein said first absorption rate calculated by said first absorption rate calculation module is written and stored, and said first absorption rate stored in the first absorption rate memory module is read out and employed operative to conduct a radiographic operation.

6. The radiographic device, according to claim 1, further comprising;
a variation rate calculation module that, calculates the variation rate of the radiation image of said subject relative to the radiation image without said subject, calculates based on the direct radiation absorption rate of each grid foil relative to the plural radiation images without said subject, calculated by said first absorption rate calculation module, and the direct radiation absorption rate of each grid foil relative to the radiation images with said subject, calculated by the second absorption rate calculation module;
wherein said distance function calculation module calculates said distance function based on said variation rate calculated by said variation calculation module.

7. The radiographic device, according to claim 6, wherein:
said scatter radiation removal module constitutes said grid foils arrayed parallel in either one direction of lengthwise or breadthwise of said radiation detection element;
the radiographic device, further comprising:
an add-value calculation module operative to calculate the add-value due to the adding or the additional averaging, wherein said variation rate calculated by said variation rate calculation module along the extending direction of the grid foils is added or averaged by an additional averaging;
the distance function calculation module calculates said distance function based on said add-value calculated by the add-value calculation module.

8. The radiographic device, according to claim 7, further comprising:
a first absorption rate memory module; and
wherein said first absorption rate calculated by said first absorption rate calculation module is written and stored, and said first absorption rate stored in the first absorption rate memory module is read out and employed operative to conduct a radiographic operation.

9. The radiographic device, according to claim 1, further comprising:
a first absorption rate memory module; and
wherein said first absorption rate calculated by said first absorption rate calculation module is written and stored, and said first absorption rate stored in the first absorption rate memory module is read out and employed operative to conduct a radiographic operation.
